(19) United States
(12) Patent Application Publication
(10) Pub. No.: US 2023/0317250 A1
(43) Pub. Date: Oct. 5, 2023

(54) NETWORK BONDING FOR MEDICAL IMAGE STREAMING FROM A MOBILE SYSTEM

(71) Applicant: Welsh Family Limited Partnership d/b/a Point of Care, Indianapolis, IN (US)

(72) Inventor: Cody Neville, Greenwood, IN (US)

(73) Assignee: Welsh Family Limited Partnership d/b/a Point of Care, Indianapolis, IN (US)

(21) Appl. No.: 18/331,439

(22) Filed: Jun. 8, 2023

Related U.S. Application Data
(63) Continuation of application No. 16/807,558, filed on Mar. 3, 2020, now abandoned.
Application No. 16/807,558 is a continuation of application No. PCT/US2018/049968, filed on Sep. 7, 2018.
(60) Provisional application No. 62/555,217, filed on Sep. 7, 2017.

(51) Int. Cl. G16H 30/20; H04W 4/30; H04L 65/80; H04W 92/02; H04L 65/70

(52) U.S. Cl. CPC G16H 30/20; H04W 4/30; H04L 65/80; H04W 92/02; H04L 65/70

(57) ABSTRACT

A system for transmitting medically-necessary video or images over a collaborative set of wireless and/or satellite networks is provided. The system utilizes a number of interfaces to one or more wireless networks and bonds these connections together to provide a reliable data stream, such as for streaming an ultrasound image from an ambulance or other remote location for viewing by a waiting surgeon or other medical professional.

### CROSS-REFERENCE TO RELATED APPLICATION

This application is a continuation of U.S. patent application Ser. No. 16/807,558 filed Mar. 3, 2020 which is a continuation of International Patent Application No. PCT/US2018/049968 filed Sep. 7, 2018 which claims the benefit of U.S. Provisional Patent Application Ser. No. 62/555,217 filed Sep. 7, 2017 entitled “NETWORK BONDING FOR MEDICAL ULTRASOUND STREAMING FROM A MOBILE SYSTEM” which is hereby incorporated by reference in its entirety to the extent not inconsistent.

### FIELD OF THE INVENTION

The present invention relates generally to reliable digital video transmission over a collaborative set of wireless and/or satellite networks. More particularly, the invention is particularly well suited for use in transmitting live or near real time medically-necessary video or images, such as an ultrasound stream, from an ambulance or other remote location for viewing by a waiting surgeon or other medical professional in a remote location.

### BRIEF DESCRIPTION OF THE DRAWINGS

FIG. 1 is a diagrammatic view of a video transmission system to an embodiment of the present disclosure.

FIG. 2A is the first portion of a flowchart showing a first portion of one set of the steps involved in transmitting live video using the transmission system of FIG. 1.

FIG. 2B is the second portion of a flowchart showing the remainder of one set of the steps involved in transmitting live video using the transmission system of FIG. 1.

FIG. 3 is a flowchart showing one set of the steps involved in receiving and reconstituting live video using the transmission system of FIG. 1.

### DESCRIPTION OF THE ILLUSTRATED EMBODIMENTS

For the purposes of promoting an understanding of the principles of the disclosure, reference will now be made to the embodiments illustrated and specific language will be used to describe the same. It will nevertheless be understood that no limitation of the scope of the disclosure is thereby intended, such alterations, modifications, and further applications of the principles being contemplated as would normally occur to one skilled in the art to which the invention relates.

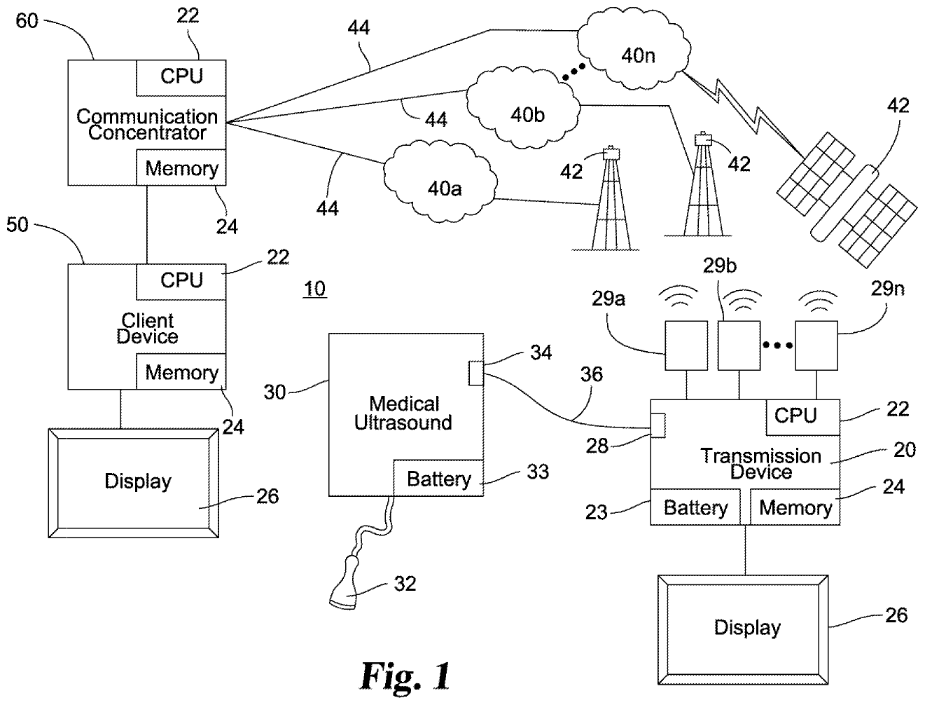

FIG. 1 is a diagrammatic view of a video transmission system 10 of one embodiment of the present invention. It shall be appreciated that the video transmission system 10 may be utilized for transmitting any type of audio and/or video where a high degree of reliability is required. In particular, the transmission system 10 is useful for transmitting live medically-necessary video or images. Exemplary devices for use with the system include an ultrasound machine, a computed tomography scan machine, a magnetic resonance imaging machine, an x-ray machine or fluoroscope machine, an endoscope or other known medical imaging devices. For purposes of illustration, and not limitation, the transmission system 10 shall be described herein as used in a medical context for transmitting an ultrasound feed in the field for evaluation by a medical professional at a remote location.

In the illustrative embodiment, transmission system 10 includes a mobile transmission bonding device/transmission device 20, medical imaging device 30, a number of wireless data networks 40a, 40b through 40n and client device 50. In some forms, transmission system 10 may also include a communication concentrator 60.

According to the illustrated embodiment, transmission device 20, client device 50 and optional communication concentrator 60 each include one or more processors or CPUs 22 and one or more types of memory 24. In one form, memory 24 is a removable memory device. Each processor 22 may be comprised of one or more components configured as a single unit. One or more components of each processor 22 may be of the electronic variety defining digital circuitry, analog circuitry, or both. In one embodiment, each processor 22 is of a conventional, integrated circuit microprocessor arrangement, such as one or more OPTERON processors supplied by ADVANCED MICRO DEVICES Corporation of One AMD Place, Sunnyvale, California 94088, USA or one or more CORE processors (including i3, i5 and i7) supplied by INTEL Corporation of 2200 Mission College Boulevard, Santa Clara, Calif. 95052, USA. Each memory 24 (removable, fixed or both) is one form of a computer-readable device. Each memory may include one or more types of solid-state electronic memory, magnetic memory, or optical memory, just to name a few examples. It shall be appreciated that the processor 22 and/or memory 24 of each of transmission device 20, client device 50 and communication concentrator 60 is not required to be of the same type or speed. In one form, transmission device 20 also includes a battery 23 capable of powering the operation of transmission device 20 for an extended period of time. In one form, battery 23 is a rechargeable battery such as one or more nickel cadmium (NiCd), nickel-metal hydride (NiMH), and lithium ion or lithium polymer batteries.

In one form, transmission device 20 and client device 50 are coupled to displays 26 and/or may include an integrated display. Although not shown to preserve clarity, transmission device 20 and client device 50 may also include one or more operator input devices such as a keyboard, mouse, track ball, light pen, and/or touch screen, to name just a few representative examples. Also, besides a display, one or more other output devices may be included such as a loudspeaker or printer. Various display and input/output device arrangements are possible. It shall be appreciated that transmission device 20, or client device 50 for that matter, may be of an alternate type, such as a mobile device, laptop or tablet utilizing the iOS, Android, Windows or any other operating system. This specifically includes iPhones and iPads (manufactured by Apple, Inc., located at 1 Infinite Loop Cupertino, CA 95014), Kindles (manufactured by Amazon.com, Inc., located at 1200 12th Avenue South, Suite 1200, Seattle, WA 98144-2734), Android phones/tablets (manufactured by various manufacturers), Surface tables (manufactured by Microsoft, Inc., located at One Microsoft Way, Redmond, Washington 98052) and other similar devices.

In addition, transmission device 20 also includes a plurality of network interfaces 29a, 29b . . . 29n. Each network interface 29 provides access to a communication network, such as communication networks 40a, 40b . . . 40n respectively. In one form, each network interface 29 interfaces with a different communication network, such as CDMA (Code Division Multiple Access), GSM (Global System for Mobiles), Long Term Evolution (LTE) or the like. In another form, two or more of network interface 29 interfaces may interface with the same communication network 40, but may effectively operate to increase bandwidth available to transmission device 20. In one form, each network interface 29 is connected to transmission device 20 via a universal serial bus (USB) connection or some other connection known in the art. In another form, one or more of network interfaces 29 is installed within transmission device 20 via a more permanent connection, such as PCI (Peripheral Component Interconnect) or the like. In yet another form, one or more of network interfaces 29 is integrated within transmission device 20. Each network interface 29 may also include a transceiver, antenna and/or satellite dish as necessary. According to one form, one or more of network interfaces 29 is a cellular communication device, such as a cellular radio, operating on a known cellular network, such as the GSM, LTE or CDMA wireless networks operated by AT&T, Verizon Wireless, Sprint, Telecom, Vodaphone or other known mobile date and/or telecommunications carriers. Alternatively, one or more of network interfaces 29 may be a satellite transceiver, such as that for use with Inmarsat's BGAN service or Iridium Communications Inc.'s satellite services.

Each communication network 40 typically includes one or more access points 42, which enable the corresponding network interface 29 to connect when in proximity thereto. Depending upon the type of network, each access point 42 may be an 802.11 compliant access point (such as for providing a Wi-fi (network), a cellular tower (for enabling Evolution-Data Optimized (EVDO), Enhanced Data rates for GSM Evolution (EDGE), 3G, 4G, LTE, WiMax, or other wireless data connection) or a satellite (for enabling satellite communication, such as that provided by Inmarsat's BGAN service). As will be appreciated, each communication network 40 couples together a number of computers (including others not shown) over network pathways 44. Communication networks 40 may further comprise a wireless or wired Local Area Network (LAN), Municipal Area Network (MAN), Wide Area Network (WAN), such as the Internet, a combination of these, or such other network arrangement as would occur to those skilled in the art. The operating logic of transmission device 20 and/or communication concentrator 60 can be embodied in signals transmitted over networks 40, in programming instructions, dedicated hardware, or a combination of these.

Transmission device 20 also includes one or more audio/video inputs, such as audio/video input 28. For purposes of non-limiting example, audio/video input 28 may include an HDMI, Mini-HDMI, Micro-HDMI, VGA, DVI-D, DVI-D, Mini-DVI, Micro-DVI, USB (or USB-C/Thunderbolt), Mini DisplayPort, composite, component or other connection known to one of skill in the art. Alternatively or additionally, a converter (not shown) may be utilized to convert the signal immediately prior to it being received by the transmission device 20 where appropriate and/or necessary. In one form, this converter may be an analog to digital converter, or the like.

Included in the illustrated form of transmission system 10 is a medical ultrasound machine 30. For purposes of background, medical ultrasound (also known as diagnostic sonography or ultrasonography) is a diagnostic imaging technique based on the application of ultrasound. It is used to see internal body structures such as tendons, muscles, joints, vessels and internal organs. Ultrasound machines, such as medical ultrasound machine 30, rely on sound waves with frequencies which are higher than those audible to humans (>20,000 Hz). Ultrasonic images also known as sonograms are made by sending pulses of ultrasound into tissue using a probe 32 which is placed into contact with the skin of the patient in the area of concern. The sound echoes off the tissue; with different tissues reflecting varying degrees of sound. These echoes are recorded and displayed as a live image/video to the operator. Many different types of images can be formed using sonographic instruments. The most well-known type is a B-mode image, which displays the acoustic impedance of a two-dimensional cross-section of tissue. Other types of image can display blood flow, motion of tissue over time, the location of blood, the presence of specific molecules, the stiffness of tissue, or the anatomy of a three-dimensional region. Compared to other prominent methods of medical imaging, ultrasound has several advantages. For instance, ultrasound provides images in real-time, it is portable, it is substantially lower in cost, and it does not use harmful ionizing radiation.

Medical ultrasound can be used to diagnose conditions or to survey the extent of injuries in the field, such as within an ambulance of medical-evacuation type scenario. However, currently the review of these images is limited to the medical technicians working in the field with the patient. By reliably providing real-time ultrasound or other medical diagnostic images to a medical professional or emergency room doctor prior to arrival of the patient, valuable time can be saved by diagnosing the patient's condition and making the necessary preparations for their arrival. Moreover, by providing the diagnostic images to a remote medical professional, a more highly skilled and/or specialized medical professional can be involved in the treatment process more rapidly, thus raising the standard of patient care.

Medical ultrasound machine 30, as shown in FIG. 1, includes a video output 34. This video output 34 may traditionally be connected to a monitor for local viewing. However, in the current form, this video output 34 is used to provide a live video feed to the video input port 28 of transmission device 20 via connection line 36. In order to provide a local view, the video feed from the medical ultrasound machine 30 may be displayed on the screen 26 of transmission device 20 and/or it may be split so as to provide the feed to some other monitor or display (not shown). In one form, medical ultrasound machine 30 also includes a battery 33 capable of powering its operation for an extended period of time. Battery 33 may be, for example, one or more of a nickel cadmium (NiCd), nickel-metal hydride (NiMH), and lithium ion or lithium polymer battery, which may be rechargeable. Battery 33 may be internal to medical ultrasound machine 30 or may be a stand-alone component for supplying power to ultrasound machine 30. Alternatively, medical ultrasound machine 30 may be powered by a portable power source, such as a generator, external battery pack or the like which may be available in the field or available within an associated vehicle, such as an ambulance.

The video transmission system 10 utilizes the transmission device 20 to transmit a single data stream, such as a digital video stream, over the plurality of included networks 40a, 40b . . . 40n, by splitting the data stream into a number of sub-parts, each of which is sent over one of the various networks, and then reassembling the sub-parts into a single coherent stream at client device 50 or alternatively at communication concentrator 60 (for viewing by client device 50).

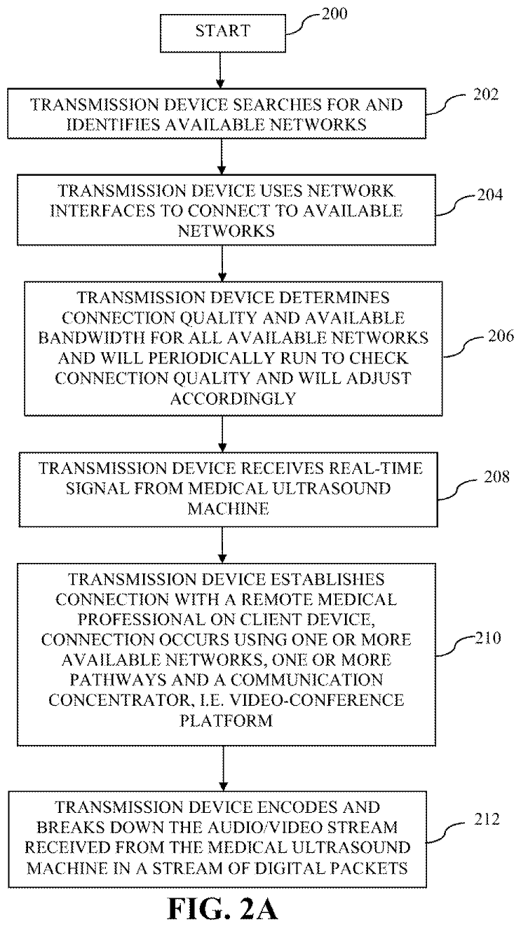

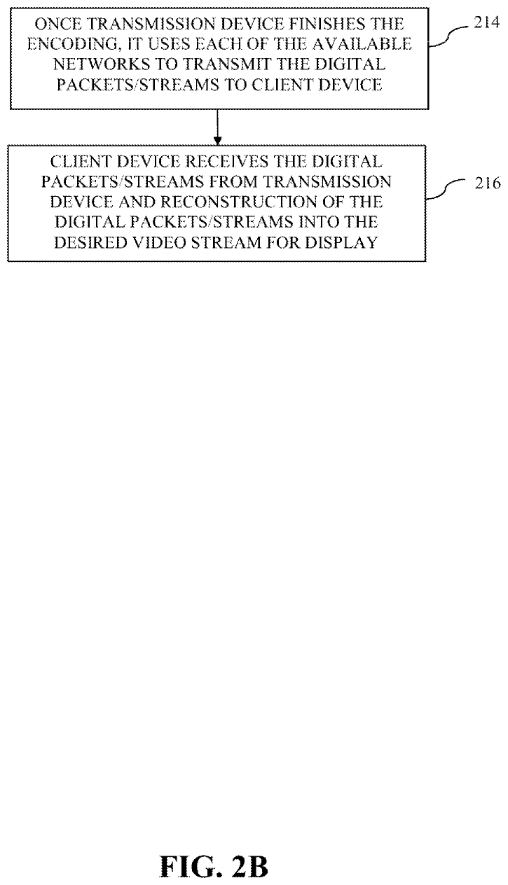

FIG. 2, which is comprised of FIGS. 2A and 2B, is a flowchart showing one set of the steps 200 involved in transmitting live or near-real time video stream using transmission system 10. The majority, if not all of the steps contained in FIG. 2, with the exception of step 216, are performed by the transmission device 10, with input and assistance from other identified components. The process begins at step 202 with the transmission device 20 searching for and identifying available networks, such as networks 40a, 40b . . . 40n. The transmission device 20, using network interfaces 29a, 29b . . . 29n, connects to the available networks 40a, 40b . . . 40n in step 204. Subsequently, in step 206, the transmission device 20 determines the quality of the connection and the available bandwidth for each available network 40a, 40b . . . 40n. As part of step 206, the transmission device stores the maximum available bandwidth, average available bandwidth and transit time for data packets sent on each data network. It shall be appreciated that step 206 may be periodically or continuously run to provide updated information and adjust for the varying reception created by the location of transmission device 20, weather, obstructions or various other known influences on wireless transmission.

In step 208, the transmission device 20 receives a real-time image or video signal, which may include audio, from the output 34 of medical ultrasound machine 30. The transmission device 20 next establishes a connection with a remote medical professional on client device 50 in step 210. This connection occurs using one or more of the available networks 40a, 40b . . . 40n, one or more of pathways 44 and optionally communication concentrator 60. It shall be appreciated that the connection may be initiated by the transmission device 20 or the client device 50, depending upon user preferences. In one particular form, the connection is established using a video-conference platform, such as that provided by WebRTC (a HIPAA compliant solution), which may require authentication, such as a username and password or the like. The video-conference connection enables bi-directional transmission of audio and video between the transmission device 20 and the client device 50.

The process continues in step 212 with the transmission device encoding and breaking down the audio/video stream received from the medical ultrasound machine 30 in a stream of digital packets. By way of example the stream may be encoded into one or more of the following types of exemplary streams: W3C, AVI, FLV, MOV, SCTP or MP4. Of course, other types of audio/video streams may be utilized depending upon user preferences. Subsequently, the transmission device 20 serializes the encoded audio/video stream into a stream of packets for transport. Once encoded and broken down, the transmission device 20 utilizes, in selective combination, each of the available networks 40a, 40b . . . 40n to transmit the stream of packets to client device 50 (step 214). This process, known as “network bonding” enables the transmission device 20 to combine the available bandwidth and throughput of the available networks 40a, 40b . . . 40n to achieve the desired and reliable stream necessary for reliable transmission, such as is required in a medical setting. Moreover, the network bonding performed by transmission device 20 extends the area where transmission device 20 may be used into areas what a given network 40 may be insufficient, due to either lack of coverage, lack of bandwidth, and/or network congestion. For example, when three networks are available, one network may receive 40% of the stream with the others each receiving 30%. Additionally, the network bonding may work in combination with the encoding processor or algorithm to periodically adjust the generated video stream to approximate the maximum quality or bitrate which can be reliably transmitted over the collective network bandwidth at a given time. For example, when bandwidth is high, a high-definition stream may be provided. However, in areas of lower quality and bandwidth, a standard definition stream may be provided at a lower framerate. Alternatively, the best network may have adequate bandwidth at a given time and it may handle 100% of the stream despite the availability of other networks. In the illustrated form, the breakdown of the stream amongst the available networks 40a, 40b . . . 40n is changed periodically as the quality of the connection and the latency of the available networks 40a, 40b . . . 40n changes. Other network criteria may also be considered. In a further form, transmission device 20 may send duplicate packets from within a single stream over the available networks 40a, 40b . . . 40n to combat the potential for packet loss and/or packet delays. In one form, the network bonding described may be accomplished using a virtual private network (VPN), such as that available from Speedify (located at 1429 Walnut St., Suite 201, Philadelphia, PA 19102 USA).

As a final step, step 216, the packets sent by transmission device 20 are received by client device 50 or communication concentrator 60 and reconstructed into the desired video stream for display.

In a further form, when two-way conferencing is enabled, the communication concentrator may perform a similar function to that described above with respect to transmission device 20 to divide the return stream amongst the available networks 40a, 40b . . . 40n for sending in the opposite direction back (i.e. back to transmission device 20). In this form, the transmission device 20 reconstitutes the stream for presentation on its attached display 26.

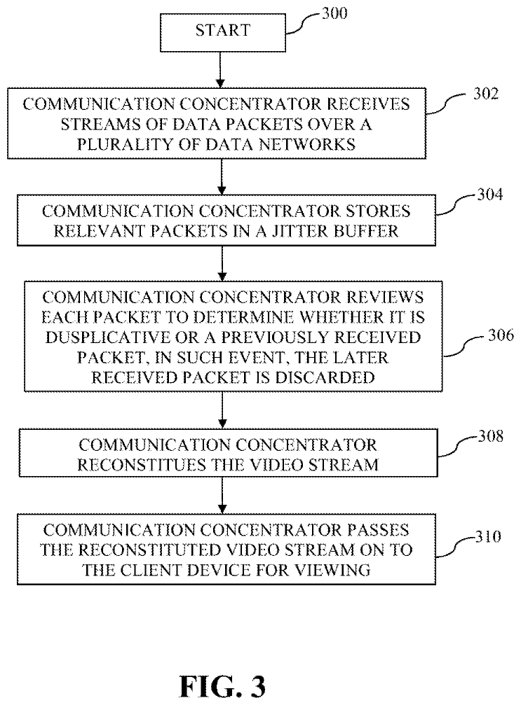

FIG. 3 is a flowchart showing one set of the steps 300 involved in reconstituting a live video stream using communication concentrator 60 of transmission system 10. It shall be appreciated that the process illustrated in FIG. 3 may be performed by client device 50 if desired, with the transmission in step 310 being unnecessary. The process begins at step 302 with the communication concentrator 60 receiving numerous distinct streams of packets which have travelled over of a plurality of data networks, such as networks 40a, 40b . . . 40n. These may be the data packets sent by the transmission device 20 in step 214 of FIG. 2. The data packets are then reviewed by the communication concentrator 60 to determine that they belong to a given stream, and the relevant packets are passed to a jitter buffer (step 304). In the event packets are duplicated and redundantly sent out by transmission device 20 over different networks 40, the communication concentrator 60 may compare each packets to those validly received before, and upon identifying a match, delete the later received packet without further consideration (step 306). In other forms, the later received packet may be utilized to correct a previously received packet which may fail a checksum or other validity verification step.

The communication concentrator 60 reorders the packets within the jitter buffer on an ongoing basis and combines them to reconstitute the video stream which was originally transmitted by the transmission device 20 (step 308) as is known by one of skill in the art. The jitter buffer parameters, such as the total time of receipt spanned by the buffer, may be adjusted by the user or automatically depending upon network conditions. In the final step 310, the reconstituted video stream is passed on by the communication concentrator 60 to the client device 50 for viewing.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

What is claimed is:

1. A communication system (10) configured to facilitate the transmission of a near real time medical image stream over a plurality of wireless communication networks (40), the communication system comprising:
a transmission bonding device (20) including a first processor (22), a video input (28) and a mobile power source (23) for supplying power to the first processor (22);
a plurality of wireless digital network interfaces (29) connected to said transmission bonding device (20), wherein each of said plurality of wireless digital network interfaces (29) is operable to provide a connection to a digital wireless data network (40);
a medical imaging device (30) having a video output (34), wherein the video output (34) is connected to and is operable to provide a video stream to the transmission bonding device (20); and
a receiving device (50) including a display (26) and a second processor (22);
wherein the first processor (22) of the transmission bonding device (20) is configured to control the plurality of wireless digital network interfaces (29) to communicate a media transmission comprising the video stream generated by the medical imaging device (30) over the digital wireless data networks (40) to the receiving device (50), such that at least a portion of the media transmission is sent over each of the wireless digital networks (40), and
wherein the second processor (22) of the receiving device (50) is configured to receive and reconstitute the portions of the media transmission and subsequently display the reconstituted media transmission on the display (26).

2. The communication system (10) of claim 1, wherein at least two of the plurality of wireless digital network interfaces (29) are configured to operate on separate and distinct digital wireless data networks (40).

3. The communication system (10) of claim 2, wherein each of the plurality of wireless digital network interfaces (29) is configured to operate on separate and distinct digital wireless data network (40) from the other wireless digital network interfaces (29).

4. The communication system of claim 10) claim 1, wherein at least two of the plurality of wireless digital network interfaces are configured to operate on the same digital wireless data network.

5. The communication system (10) of claim 2, wherein at least one of the plurality of wireless digital network interfaces (29) is configured to operate on a Code Division Multiple Access (CDMA) type digital wireless data network (40).

6. The communication system (10) of claim 2, wherein at least one of the plurality of wireless digital network interfaces (29) is configured to operate on a Global System for Mobiles (GSM) type digital wireless data network (40).

7. The communication system (10) of claim 2, wherein at least one of the plurality of wireless digital network interfaces (29) is configured to operate on a Long Term Evolution (LTE) type digital wireless data network (40).

8. The communication system (10) of claim 2, wherein at least one of the plurality of wireless digital network interfaces (29) is configured to operate on a direct to satellite type data network (40).

9. The communication system (10) of claim 1, wherein at least one of the plurality of wireless digital network interfaces (29) is connected to the transmission bonding device (20) via a universal serial bus (USB) connection.

10. The communication system (10) of claim 1, wherein the mobile power source (23) is a rechargeable battery.

11. The communication system (10) of claim 10, wherein the mobile power source (23) is integrated within transmission bonding device (20).

12. The communication system (10) of claim 1, wherein the video stream contains near real time medical images.

13. The communication system (10) of claim 12, wherein the medical device (30) is an ultrasound machine.

14. The communication system (10) of claim 12, wherein the medical device (30) is a computed tomography scan machine.

15. The communication system (10) of claim 12, wherein the medical device (30) is a magnetic resonance imaging device.

16. The communication system (10) of claim 12, wherein the medical device (30) is an x-ray machine.

17. The communication system (10) of claim 12, wherein the transmission bonding device (20) is mounted within an ambulance.

18. The communication system (10) of claim 2, wherein the first processor (22) of the transmission bonding device (20) is further configured to control the plurality of wireless digital network interfaces (29) to communicate at least a portion of the media transmission comprising the video stream generated by the medical imaging device (30) redundantly over at least two of the wireless digital networks (40).

19. A communication system (10) configured to facilitate the transmission of a near real time medical image stream over a plurality of wireless communication networks (40), the communication system (10) comprising:
a transmission bonding device (20) including a first processor (22), a video input (28) and a mobile power source (23) for supplying power to the first processor (22);
a plurality of wireless digital network interfaces (29) connected to said transmission bonding device (20), wherein each of said plurality of wireless digital network interfaces (29) is operable to provide a connection to a digital wireless data network (40);
a medical imaging device (30) having a video output (34), wherein the video output (34) is connected to and is operable to provide a video stream to the transmission bonding device (20); and
a communication concentrator (60) including a second processor (22);
a receiving device (50) including a third processor (22) and a display (26);
wherein the first processor (22) of the transmission bonding device (20) is configured to control the plurality of wireless digital network interfaces (29) to communicate a media transmission comprising the video stream generated by the medical imaging device (30) over the digital wireless data networks (40) to the communication concentrator (60), such that at least a portion of the media transmission is sent over each of the wireless digital networks (40), and
wherein the second processor (22) of the communication concentrator (60) is configured to receive and reconstitute the portions of the media transmission and transmit the reconstituted media transmission to the receiving device (50); and
wherein the third processor (22) of the receiving device (50) is configured to receive the reconstituted media transmission from the communication concentrator (60) and subsequently display the reconstituted media transmission on the display (26).

20. A communication system (10) configured to facilitate the transmission of a near real time ultrasound medical image stream over a plurality of wireless communication networks (40), the communication system (10) comprising:
a transmission bonding device (20) including a first processor (22), a video input (28) and a rechargeable battery (23) for supplying power to the first processor (22);
a plurality of wireless digital network interfaces (29) connected to said transmission bonding device (20), wherein each of said plurality of wireless digital network interfaces (29) is operable to provide a connection to a digital wireless data network (40);
an ultrasound medical imaging device (30) having a video output (34), wherein the video output (34) is connected to and is operable to provide a video stream to the transmission bonding device (20); and
a receiving device (50) including a display (26) and a second processor (22);
wherein the first processor (22) of the transmission bonding device (20) is configured to control the plurality of wireless digital network interfaces (29) to communicate a media transmission comprising the video stream generated by the medical imaging device (30) over the digital wireless data networks (40) to the receiving device (50), such that at least a portion of the media transmission is sent over each of the wireless digital networks (40), and
wherein the second processor (22) of the receiving device (50) is configured to receive and reconstitute the portions of the media transmission and subsequently display the reconstituted media transmission on the display (26).
